(19) United States
(12) Patent Application Publication
(10) Pub. No.: US 2025/0245841 A1
(43) Pub. Date: Jul. 31, 2025

(54) SUBJECT TRACKING METHOD FOR MEDICAL IMAGING AND MEDICAL IMAGING SYSTEM

(71) Applicant: GE Precision Healthcare LLC, Waukesha, WI (US)

(72) Inventors: Bin Xie, Beijing (CN); Ting Ye, Beijing (CN)

(21) Appl. No.: 19/034,184

(22) Filed: Jan. 22, 2025

(30) Foreign Application Priority Data
Jan. 29, 2024 (CN) 202410122613.6

(51) Int. Cl. G06T 7/246; G06T 7/73

(52) U.S. Cl. CPC G06T 7/248; G06T 7/74; G06T 2200/24; G06T 2207/30196

(57) ABSTRACT

A subject tracking method for medical imaging is provided. The method includes acquiring a plurality of frames of image data containing a subject, which are captured via an image capture apparatus; detecting information of anatomical key points in each of the plurality of frames of image data; and determining a state of the subject based on the information of the anatomical key points.

### CROSS REFERENCE TO RELATED APPLICATIONS

The present application claims priority and benefit of Chinese Patent Application No. 202410122613.6 filed on Jan. 29, 2024, which is incorporated herein by reference in its entirety.

### TECHNICAL FIELD

Embodiments of the present application relate to the technical field of medical devices, and in particular, to a subject tracking method for medical imaging, and a medical imaging system.

### BACKGROUND

Medical imaging of a subject can be performed by various means, including but not limited to a computed tomography (CT) system, a magnetic resonance imaging (MRI) system, etc. When undergoing a scan for said medical imaging, the subject needs to enter a scan room, while an operator needs to enter an operating room to control a medical imaging system. As a result, the operator cannot monitor the subject's position or posture within the scan room in real time, and consequently cannot start or stop scan-related processing promptly, and even cannot identify safety accidents happening to the subject in a timely manner.

### SUMMARY

Embodiments of the present application provide a subject tracking method for medical imaging, and a medical imaging system.

According to one aspect of the embodiments of the present application, a subject tracking method for medical imaging is provided. The method includes: acquiring a plurality of frames of image data containing a subject, which are captured via an image capture apparatus and detecting information of anatomical key points in each of the plurality of frames of image data. The method also includes determining a state of the subject based on the information of the anatomical key points.

According to one aspect of the embodiments of the present application, a computer-readable storage medium is provided. The computer-readable storage medium includes a stored computer program, and the subject tracking method for medical imaging described in the foregoing aspect is executed when the computer program is run.

According to one aspect of the embodiments of the present application, a medical imaging system is provided. The system includes: an image capture apparatus, capturing a plurality of frames of image data containing a subject and a controller, connected to the image capture apparatus and used to execute the subject tracking method in the foregoing aspect.

With reference to the following description and drawings, specific implementations of the embodiments of the present application are disclosed in detail, and the means by which the principles of the embodiments of the present application can be employed are illustrated. It should be understood that the embodiments of the present application are not limited in scope thereby. Within the scope of the spirit and clauses of the appended claims, the embodiments of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of a magnetic resonance imaging system according to embodiments of the present application;

FIG. 2 is a schematic diagram of a subject tracking method for medical imaging according to embodiments of the present application;

FIG. 3 is a schematic diagram of a medical imaging system according to embodiments of the present application;

FIG. 4 is a schematic diagram of a magnetic resonance imaging system according to embodiments of the present application;

FIG. 5 is a schematic diagram of a subject tracking apparatus for medical imaging according to embodiments of the present application;

FIG. 6 is a schematic diagram of a subject tracking method for medical imaging according to embodiments of the present application;

FIG. 7 is a schematic diagram of a medical imaging method according to embodiments of the present application;

FIG. 8 is a schematic diagram of anatomical key points according to embodiments of the present application;

FIG. 9 is a schematic diagram of a user interface according to embodiments of the present application; and

FIG. 10 is a schematic diagram of a scanning method according to embodiments of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

A medical imaging system described herein includes, but is not limited to, a computed tomography (CT) system, a magnetic resonance imaging (MRI) system, a C-arm imaging system, a positron emission computed tomography (PET) system, a single photon emission computed tomography (SPECT) system, an ultrasound system, an X-ray imaging system, or any other suitable medical imaging system.

In the following, a magnetic resonance imaging system is used as an example for description, but embodiments of the present application are not limited thereto.

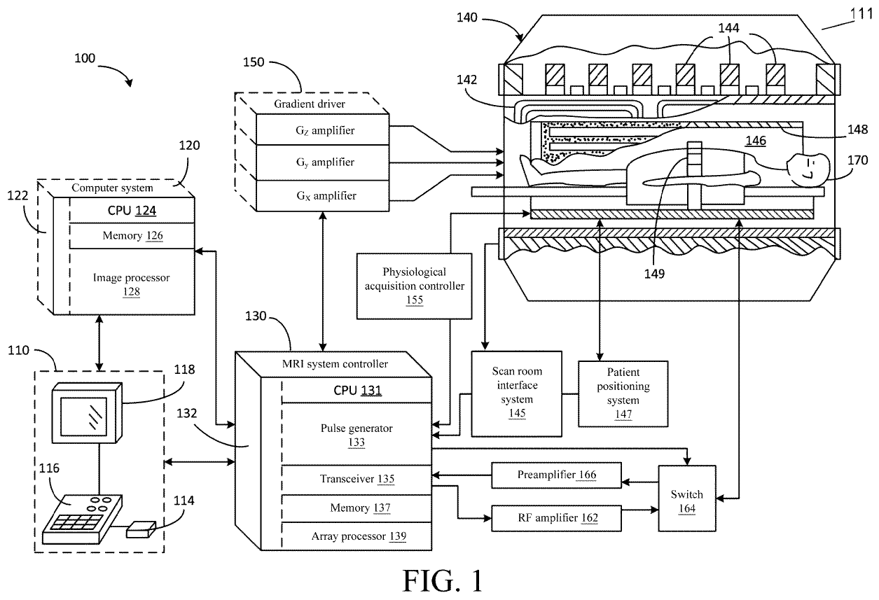

For ease of understanding, FIG. 1 shows a magnetic resonance imaging (MRI) system 100 according to some embodiments of the present invention.

The MRI system 100 includes a scanning unit 111. The scanning unit 111 is used to perform a magnetic resonance scan on a subject (for example, a human body) 170 to generate a reconstructed image of a region of interest of the subject 170. The region of interest may be a predetermined anatomical site or anatomical tissue.

The operation of the MRI system 100 is controlled by an operator workstation 110 that includes an input device 114, a control panel 116, and a display 118. The input apparatus 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and in communication with a computer system 120 that enables an operator to control the generation and display of images on the display 118. The computer system 120 includes various components that communicate with one another via an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced by image processing functions implemented in the CPU 124. The computer system 120 may be connected to an archive media device, a persistent or backup memory, or a network. The computer system 120 may be coupled to and communicates with a separate MRI system controller 130.

The MRI system controller 130 includes a set of components that communicate with one another via an electrical and/or data connection module 132. The connection module 132 may employ a direct wired connection, a fiber optic connection, a wireless communication link, etc. The MRI system controller 130 may include a CPU 131, a sequence pulse generator 133 which is in communication with the operator workstation 110, a transceiver (or an RF transceiver) 135, a memory 137, and an array processor 139. In some embodiments, the sequence pulse generator 133 may be integrated into a resonance assembly 140 of the scanning unit 111 of the MRI system 100. The MRI system controller 130 may receive a command from the operator workstation 110, and is coupled to the scanning unit 111 to indicate an MRI scanning sequence to be performed during an MRI scan, so as to be used to control the scanning unit 111 to perform the flow of the aforementioned magnetic resonance scan. The MRI system controller 130 is further coupled to and in communication with a gradient driver system 150, which is coupled to a gradient coil assembly 142 to generate a magnetic field gradient during the MRI scan.

The sequence pulse generator 133 may further receive data from a physiological acquisition controller 155, which receives signals from a number of different sensors, such as electrocardiogram (ECG) signals from electrodes attached to a patient, which are connected to the subject or patient 170 undergoing an MRI scan. The sequence pulse generator 133 is coupled to and in communication with a scan room interface system 145 that receives signals from various sensors associated with the state of the resonance assembly 140. The scan room interface system 145 is further coupled to and in communication with a patient positioning system 147 that sends and receives signals to control movement of a patient table to a desired position to perform the MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, so as to generate a magnetic field gradient used to spatially encode an MR signal during an MRI scan. The gradient coil assembly 142 is disposed within the resonance assembly 140, and the resonance assembly further includes a superconducting magnet having a superconducting coil 144 that, in operation, provides a static uniform longitudinal magnetic field B₀ throughout a cylindrical imaging volume 146. The resonance assembly 140 further includes an RF body coil 148, which, in operation, provides a transverse magnetic field B₁, the transverse magnetic field B₁ being substantially perpendicular to B₀ throughout the entire cylindrical imaging volume 146. The resonance assembly 140 may further include an RF surface coil 149 for imaging different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in the k-space. The y direction may be referred to as a phase encoding direction or a k_{y} direction in the k-space. Gₓ can be used for frequency encoding or signal readout, and is generally referred to as a frequency encoding gradient or a readout gradient. G_{y} can be used for phase encoding, and is generally referred to as a phase encoding gradient. G_{z} can be used for slice (layer) position selection to obtain k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses that are amplified by an RF amplifier 162 and provided to the RF body coil 148 through a transmit/receive switch (T/R switch) 164.

As described above, the RF body coil 148 and the RF surface coil 149 may be used to transmit RF excitation pulses and/or receive resulting MR signals from the patient undergoing the MRI scan. The MR signals emitted by excited nuclei in the patient of the MRI scan may be sensed and received by the RF body coil 148 or the RF surface coil 149 and sent back to a preamplifier 166 through the T/R switch 164. The T/R switch 164 may be controlled by a signal from the sequence pulse generator 133 to electrically connect the RF amplifier 162 to the RF body coil 148 in the transmit mode and to connect the preamplifier 166 to the RF body coil 148 in the receive mode. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are stored in the memory 137 for post-processing as a raw k-space data array. A reconstructed magnetic resonance image may be obtained by transforming/processing the stored raw k-space data.

In some embodiments, the MR signals sensed and received by the RF body coil 148 or the RF surface coil 149 and amplified by the preamplifier 166 are demodulated, filtered, and digitized in a receiving portion of the transceiver 135, and transmitted to the memory 137 in the MRI system controller 130. For each image to be reconstructed, the data is rearranged into a separate k-space data array, each of said separate k-space data arrays is input into the array processor 139, and the array processor is operated to transform the data into a reconstructed image by Fourier transform.

The array processor 139 uses transform methods, most commonly Fourier transform, to create images from the received MR signals. These images are transmitted to the computer system 120 and stored in the memory 126. In response to commands received from the operator workstation 110, data for the reconstructed image may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or collectively include a computer processor and a storage medium. The storage medium records a predetermined data processing program to be executed by the computer processor. For example, the storage medium may store a program used to implement scanning processing (such as a scan flow and an imaging sequence), image reconstruction, image processing, etc. For example, the storage medium may store a program used to implement the magnetic resonance imaging method according to the embodiments of the present invention. The described storage medium may include, for example, a ROM, a floppy disk, a hard disk, an optical disk, a magneto-optical disk, a CD-ROM, or a non-volatile memory card.

The aforementioned “imaging sequence” (also referred to below as a scanning sequence or a pulse sequence) refers to a combination of pulses having specific amplitudes, widths, directions, and time sequences and applied when a magnetic resonance imaging scan is executed. These pulses may typically include, for example, radio-frequency pulses and gradient pulses. The radio-frequency pulses may include, for example, radio-frequency excitation pulses, radio-frequency refocusing pulses, inverse recovery pulses, etc. The gradient pulses may include, for example, the aforementioned gradient pulse used for layer selection, gradient pulse used for phase encoding, gradient pulse used for frequency encoding, gradient pulse used for phase shifting (phase shift), gradient pulse used for dispersion of phases (dephasing), etc. Typically, a plurality of scan sequences can be preset in the magnetic resonance system, so that the sequence suitable for clinical detection requirements can be selected. The clinical detection requirements may include, for example, an imaging site, an imaging function, an imaging effect, and the like.

Currently, in a medical imaging system (e.g., a magnetic resonance imaging system or a computed tomography imaging system), an image capture apparatus may be introduced to obtain visual and morphological information of a human body. The image capture apparatus may be mounted near an examination bed to maximally acquire auxiliary information of a subject in a non-contact manner. Acquired images can be used to assist in medical imaging operations, such as automatic positioning, automatic identification of a subject's orientation, etc.

However, one may find that a subject in a scan room may get up at any time to adjust a pose, leave a scanning bed, fall from the scanning bed, etc. However, after an operator enters an operating room and is operating a medical imaging system to perform processing such as positioning or orientation identification, the operator likely cannot detect a change in a state of the subject in time, resulting in a failure in the work flow or even an accident.

In addition, one may find that, although many tracking methods are currently available in the field of computer vision, none of the methods can be easily used directly in medical imaging systems. For example, in some scenarios, it is necessary to cover a surface of a subject with an occluding object (e.g., a coil, a blanket, complex clothes, or a mask). As a result, existing tracking methods always lose track of the subject as some regions of the subject are covered with the occluding object.

In view of at least one of the foregoing technical problems, embodiments of the present application provide a subject tracking method for medical imaging. A state of a subject is determined based on anatomical key points identified in images captured by an image capture apparatus. In this way, automatic tracking can be implemented after the subject enters a scan room, so that the determined state of the subject can be used as a condition for triggering other procedures in a scanning process, e.g., automatic positioning, automatic identification of an orientation, user interface display, execution of an occlusion algorithm, etc., thereby implementing further automation of the scanning process and improving scanning efficiency. In addition, when it is detected that the subject gets up, gets out of bed or falls, a running procedure can be stopped in time. For example, the procedures for automatic positioning, automatic identification of an orientation, user interface display, and the execution of the occlusion algorithm can be stopped, thereby avoiding faulty execution of the procedures. In addition, an alert may be further issued to warn the clinical operator.

Description is made below in conjunction with the embodiments.

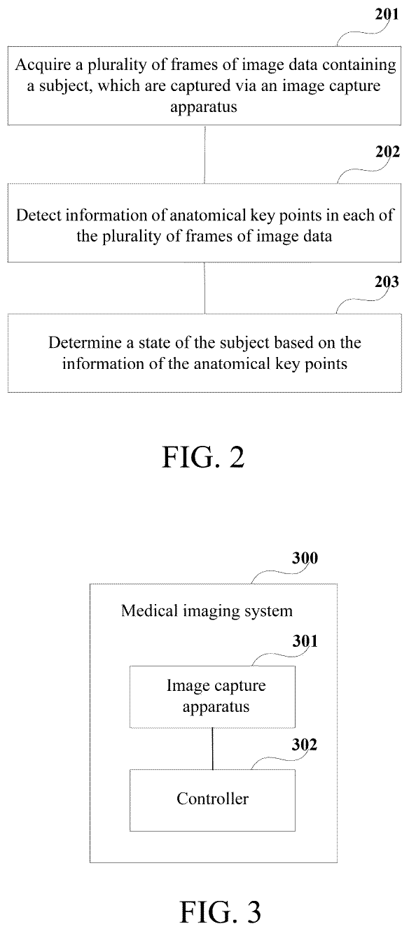

Embodiments of the present application provide a subject tracking method for medical imaging. FIG. 2 is a schematic diagram of the subject tracking method for medical imaging according to the embodiments of the present application. As shown in FIG. 2, the method includes a step 201 for acquiring a plurality of frames of image data containing a subject that are captured via an image capture apparatus and a step 202 for detecting information of anatomical key points in each of the plurality of frames of image data. The method also includes a step 203 for determining a state of the subject based on the information of the anatomical key points.

In some embodiments, the image data of the subject may be acquired by the image capture apparatus. The image data may include, but is not limited to, at least one of two-dimensional optical image data and depth image data. For example, the image capture apparatus may be a 3D camera. The 3D camera may capture the whole body of the subject in real time, to generate a video stream, each frame in the video stream including the two-dimensional optical image and the depth image. The two-dimensional optical image may be a two-dimensional RGB image, and each pixel value of the depth image reflects a distance between the camera and a corresponding position of the subject. The image capture apparatus may be mounted at a ceiling or a wall directly above an examination bed of a medical imaging system. Embodiments of the present application are not limited thereto.

In the above, the image capture apparatus being a 3D camera is used as an example, but the present application is not limited thereto. For example, the two-dimensional optical image data and the depth image data may also be obtained by a separate 2D optical camera and a separate depth camera, respectively. No further examples will be provided herein.

In some embodiments, the plurality of frames of image data may be a plurality of continuous frames of image data or a plurality of discontinuous frames of image data (for example, adjacent frames being spaced apart by an interval of a number X of frames) within a predetermined time. For example, the predetermined time may be 1 minute or 5 minutes, etc., and the plurality of frames of image data may be image data of an N^{th} frame, an (N+1)^{th} frame, . . . , and an (N+M)^{th} frame in the video stream. That is, the quantity of frames in the plurality of frames of image data is M, M being an integer greater than or equal to 2. The embodiments of the present application are not limited thereto.

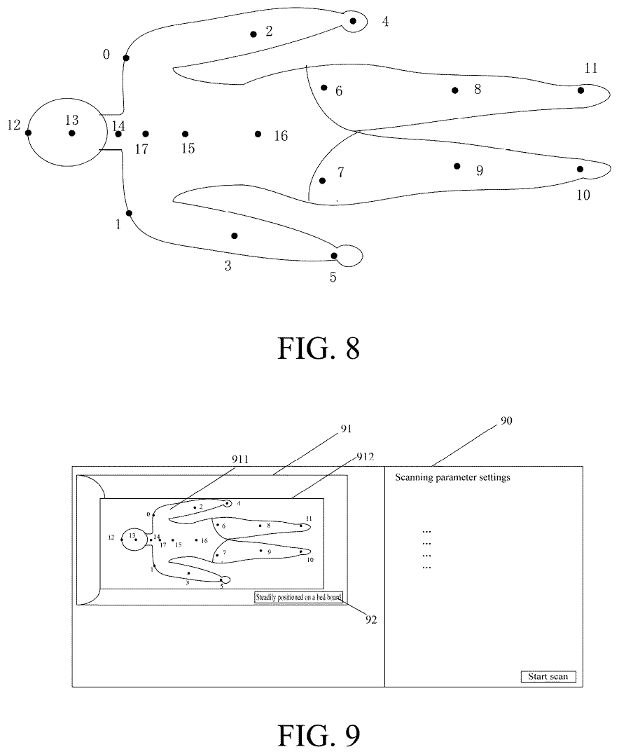

In some embodiments, in 202, each frame of image data may be separately detected using a depth learning algorithm, so as to identify the anatomical key points in each frame of image data. The quantities and types of the anatomical key points in the frames of image data are the same or different. The anatomical key points include: at least two of the top of the head, a shoulder, the nose, an eye, an ear, an arm, an elbow, a wrist, a hip, a knee, the heart, a pelvic cavity, the abdomen, the chest, and an ankle. As shown in FIG. 8, anatomical key points 1 to 16 are detected.

For example, a human body pose estimation model may be used to extract the anatomical key points from the image data. The human body pose estimation model is implemented based on a deep learning algorithm. For details, references may be made to related technologies. For example, a densepose model, an openpose model, an HRNet model, etc. are used to extract the described anatomical key points from the image data. Image data of a plurality of volunteers that is acquired in advance may be used as an input parameter set, and a plurality of pre-calibrated anatomical key points corresponding to each piece of image data may be used as an output parameter set. The human body pose estimation model is trained using the input parameter set and the output parameter set, and the trained human body pose estimation model is used to extract the anatomical key points.

In some embodiments, after the anatomical key points are identified, the information of the anatomical key points is determined. The information of the anatomical key points includes at least one of the following types of information: anatomical types of the key points, positional coordinates of the key points, confidence levels of the key points, and depth information of the key points.

For example, the positional coordinates of the key points may be coordinates (two-dimensional pixel coordinates) of the anatomical key points in an image coordinate system, or may be three-dimensional spatial coordinates of a camera coordinate system or three-dimensional spatial coordinates of a medical imaging coordinate system. For coordinate conversion, reference may be made to related technologies, and details are not described herein.

For example, the anatomical types of the key points include the described anatomical sites of the top of the head, the shoulder, the nose, the eye, the ear, the arm, the elbow, the wrist, the hip, the knee, the heart, the pelvic cavity, the abdomen, the chest, and the ankle, etc.

For example, the confidence levels of the key points may be represented in the form of probabilities, e.g., the probabilities that actual positions of the key points are detected positions. The present application is not limited thereto, and the confidence level information of the key points may be in other forms or has other definitions.

For example, the depth information of the key points includes depth values of the key points, represented by values of distances between pixel positions of the key points and a reference plane (e.g., a camera).

The described anatomical types, coordinates, and confidence levels may all be output parameters of the described human body pose estimation model, and will be output together by the human body pose estimation model after the two-dimensional optical image data is input into the human body pose estimation model. When the human body pose estimation model obtains the coordinates of the key points by estimation, depth values of coordinate positions corresponding to the coordinate positions are determined from the depth images as the depth information of the key points.

In some embodiments, information I of the described anatomical key points is determined for each frame of image data, and information of the anatomical key points in the plurality of frames of image data is also obtained. The information of the anatomical key points may be arranged in time order, and successively correspond to each frame of image data. For example, the information of the anatomical key points of the plurality of frames of image data may be represented as follows:

$${{I}_{1}}^{N} , {{I}_{2}}^{N} , {{I}_{3}}^{N} , {{I}_{4}}^{N} , … , and {{I}_{A}}^{N}$$

$${{I}_{1}}^{N + 1} , {{I}_{2}}^{N + 1} , {{I}_{3}}^{N + 1} , {{I}_{4}}^{N + 1} , … , and {{I}_{B}}^{N + 1}$$

$${{I}_{1}}^{N + 2} , {{I}_{2}}^{N + 2} , {{I}_{3}}^{N + 2} , {{I}_{4}}^{N + 2} , … , and {{I}_{C}}^{N + 2}$$

$$…$$

$${{I}_{1}}^{N + M} , {{I}_{3}}^{N + M} , {{I}_{5}}^{N + M} , … , and {{I}_{D}}^{N + M}$$

The subscript of I may represent the type of a key point. As shown in FIGS. 8, 0 and 1 represent the shoulders, 2 and 3 represent the elbows, 4 and 5 represent the wrists, 6 and 7 represent the pelvis, 8 and 9 represent the knees, 10 and 11 represent the ankles, 12 represents the head top, 13 represents the nose, 14 represents the neck, 16 represents the abdomen, and 15 and 17 represent the chest. Anatomical key points detected in one frame of image data may be arranged in ascending order of the numerals representing the types of the key points. If information of an anatomical key point corresponding to a numeral is not included, it indicates that the corresponding anatomical key point is not detected in that frame of image data. For example, the left elbow and the left wrist of the subject are not detected in the (N+M)^{th} frame of image.

In some embodiments, the state of the subject may be determined based on all detected anatomical key points, but the embodiments of the present application are not limited thereto. Alternatively, an anatomical key point having a confidence level higher than a threshold (e.g., 60%) may be selected from the detected anatomical key points; and the state of the subject may be determined based on information of the selected anatomical key point, thereby improving accuracy in detecting the state of the subject.

In some embodiments, the state of the subject that may be detected includes at least one of: whether the subject is moving, a moving direction of the subject, a position of the subject when not moving, whether the subject is partially occluded, and a pose of the subject (getting up, falling, lying down, etc.), which are described separately below.

In some embodiments, in 203, a reference position of the subject in each frame of image data may be determined based on the information of the anatomical key points; and it is determined, based on the reference positions in the plurality of frames of image data, that the state of the subject is “no movement” or “moving”.

In the embodiments, the reference position is determined with respect to the information of the anatomical key points in each frame of image data. The reference position may be a center-of-gravity position, or a center position of the upper limbs, or a head position, or a center position of the lower limbs, etc., i.e., determined based on the anatomical key points. The embodiments of the present application are not limited thereto. For example, for each frame of image data, average values of positional coordinates of a plurality of anatomical key points (all anatomical key points or anatomical key points having confidence levels higher than the threshold, which will not be repeated below) are calculated, or a clustering algorithm is executed on positional coordinates of some anatomical key points (for example, a clustering algorithm for anatomical key points in the upper body, to calculate the center position of the upper limbs), or coordinates of an anatomical key point of the head are selected to obtain image coordinates or three-dimensional spatial coordinates in the camera coordinate system or three-dimensional spatial coordinates of the medical imaging coordinate system for the foregoing reference position. The reference positions are arranged in time order, and successively correspond to each of the described plurality of frames of image data. For example, a reference position R^{N} is determined with respect to the information I₁^{N}, I₂^{N}, I₃^{N}, I₄^{N}, . . . , and I_{A}^{N} of the anatomical key points in the N^{th} frame of image data, a reference position R^{N+1} is determined with respect to the information I₁^{N+1}, I₂^{N+1}, I₃^{N+1}, I₄^{N+1}, . . . , and I_{B}^{N+M} of the anatomical key points in the (N+1)^{th} frame of image data, and by analogy, a reference position R^{N+M} is determined with respect to the information I₁^{N+M, I}₂^{N+M}, I₃^{N+M}, I₄^{N+M}, . . . , and I_{D}^{N+M} of the anatomical key points in the (N+M)^{th} frame of image data. The reference positions of the plurality of frames of image data may be represented in time order as follows: R^{N}, R^{N+1}, R^{N+2}, . . . , and R^{N+M}.

In the embodiments, after the reference position in each frame of image data is determined, the reference position may be tracked to determine a change trend of the coordinates of the reference position over time, and it is determined, based on the change trend of the coordinates of the reference position over time, that the state of the subject is “no movement” or “moving”. For example, if the coordinates of the plurality of reference positions arranged in time order remain unchanged or a change range is within a preset range, it is determined that the subject is not moving; or if the coordinates of the plurality of reference positions arranged in time order change or a change range exceeds a preset range, it is determined that the subject is moving.

In some embodiments, optionally, based on at least one of a result of comparison between a standard position and the reference positions in the plurality of frames of image data, or based on depth information of the reference positions in the plurality of frames of image data, a moving direction of the subject or a position of the subject when not moving may also be determined.

In the embodiments, when it is determined, based on the reference positions, that the subject is not moving, the reference positions and a standard position (e.g., a coordinate position of a bed board carrying the subject) in the same coordinate system may be further compared. If the reference positions are within a range of the standard position, it is determined that the subject is steadily positioned on the bed board.

In the embodiments, when it is determined, based on the reference positions, that the subject is moving, a movement trajectory of the subject may be obtained based on coordinate changes of the plurality of reference positions arranged in time order. When the initial reference position R^{N} in time order is compared with a standard position (e.g., a coordinate position of a bed board carrying the subject) in the same coordinate system, if the initial reference position is not within a range of the standard position but a trajectory of the initial reference position gradually approaches the standard position, it is determined that the moving direction of the subject is moving towards the bed board. If the initial reference position is within the range of the standard position but the trajectory of the initial reference position is gradually away from the standard position, it is determined that the moving direction of the subject is moving away from the bed board.

In the embodiments, when it is determined, based on the reference positions, that the subject is moving, the moving direction of the subject may be further determined based on the depth information of the reference positions in the plurality of frames of image data. When the coordinates of the reference positions are obtained, depth values of coordinate positions corresponding to the coordinate positions are determined from the depth images as the depth information of the reference positions. With respect to the depth values of the plurality of reference positions arranged in time order, if it is determined that the depth values of the reference positions are getting closer to a depth value of a standard position (e.g., a coordinate position of a bed board carrying the subject), it is determined that the moving direction of the subject is moving towards the bed board. If it is determined that the differences of depth values of the reference positions and a depth value of a standard position (e.g., a coordinate position of a bed board carrying the subject) continually increase, it is determined that the moving direction of the subject is moving away from the bed board. Alternatively, if the depth values of the reference positions continually increase, it is determined that the moving direction of the subject is moving away from the bed board; or if the depth values of the reference positions continually decrease, it is determined that the moving direction of the subject is moving towards the bed board.

In some embodiments, in 203, it may be determined, based on a change in the quantity of anatomical key points and a change in positions of the anatomical key points in the plurality of frames of image data, whether the state of the subject is “partially occluded”. With respect to the quantity of the anatomical key points in the plurality of frames of image data arranged in time order, if the quantity of the anatomical key points continually decreases over time, and the coordinates of remaining anatomical key points that have not lost remain unchanged (or change within a preset range), it is determined that the state of the subject is “partially occluded”. If the quantity of the anatomical key points increases over time and anatomical key points apart from newly added anatomical key points have unchanged coordinates, it is determined that the state of the subject is “partial occlusion removed”. To improve detection accuracy, when it is determined that the state of the subject is “no movement” and the subject has been steadily positioned on a bed board, it may be detected whether the subject is partially occluded, but the embodiments of the present application are not limited thereto.

In some embodiments, in 203, a change in pose of the subject may be determined based on at least one of a change in positions of the anatomical key points in the plurality of frames of image data, a change in a distance between anatomical key points, and a change in depths of the anatomical key points. The pose change includes but is not limited to “getting up”, “lying down”, “falling”, etc. The following provides a description by using “getting up”, “lying down” and “falling” as examples.

In the embodiments, it may be determined, based on a change in distance between anatomical key points, that the state of the subject is “getting up” or “lying down”. The distance may be a distance in an image coordinate system, a camera coordinate system, or a medical imaging coordinate system, and the embodiments of the present application are not limited thereto. For example, based on coordinates of the head and the pelvic cavity in the anatomical key points, a distance between the head and the pelvic cavity in the same frame of image is calculated. With respect to distances calculated for the plurality of frames of image data arranged in time order, a change trend of the distance over time is determined. If the distance decreases over time, it is determined that the subject is getting up, and is changing from a lying position to a sitting position. If the distance increases over time, it is determined that the subject is lying down, and is changing from a sitting position to a lying position.

In the embodiments, it may be determined, based on the change in the depths of the anatomical key points, that the state of the subject is “getting up” or “lying down”. For example, depth information of the head in the anatomical key points is determined. A change trend of the depth information over time is determined according to the depth information of the head in the plurality of frames of image data arranged in time order. If the change trend of the depth information of the head is approaching the image capture apparatus (for example, depth values decrease), it is determined that the subject is getting up and is changing from a lying position to a sitting position. If the change trend of the depth information of the head is moving away from the image capture apparatus (for example, depth values increase), it is determined that the subject is lying down and is changing from a sitting position to a lying position.

In the embodiments, it may be determined, based on the change in the positions of the anatomical key points in the plurality of frames of image data, whether the state of the subject is “falling”. For example, according to the coordinates of the anatomical key points in the plurality of frames of image data arranged in time order, if the position coordinates of all anatomical key points, in the plurality of frames of image data, used to determine the state of the subject change from being within the bed board position region to being outside the bed board position region, and a predetermined proportion (for example, 60%) or more of the anatomical key points have unchanged coordinate positions (or coordinate positions that change within a preset range) after moving outside the bed board position region, it is determined that the subject has fallen from the bed board.

The various means of detecting the state of the subject above may be implemented separately or in combination, and the embodiments of the present application are not limited thereto. For example, when the moving direction of the subject is determined, the state of the subject may be determined based on a combination of the coordinates of the reference positions and a change in depth values. Only when a state of the subject determined based on the position coordinates is the same as a state of the subject determined based on the change in the depth values, can this identical state be used as a detected state of the subject. No further examples will be provided here.

In addition, at least one of the foregoing determination means may be performed separately for a plurality of sets of M-frame image data in the video stream obtained by the image capture apparatus. For example, after the subject enters a scan room, the N^{th} to (N+M)^{th} frames of image data are obtained first, and the state of the subject is determined based on detected anatomical key points; then the (N+M+1)^{th} to the (N+2M)^{th} frames of image data are acquired, and the state of the subject is determined based on detected anatomical key points, and so on, until the subject leaves the scan room.

In some embodiments, after the state of the subject is determined, at least one of triggering a display change of a user interface of a medical imaging system, triggering automatic positioning processing, triggering processing for automatically identifying an orientation of the subject, triggering a blanket and coil occlusion detection algorithm, or issuing an alert.

As an example, after the state of the subject is determined, a display change of a user interface of a medical imaging system is triggered. For example, when it is determined that the state of the subject is “moving”, “lying down”, “partially occluded” or “steadily positioned on a bed board”, the user interface on a display of the medical imaging system is automatically triggered to start to display detected anatomical key points of the subject. FIG. 9 is a schematic diagram of a user interface according to embodiments of the present application. As shown in FIG. 9, the user interface 90 includes a display window 91 for images captured by the image capture apparatus. When it is determined that the state of the subject is “lying on a bed board 912”, the display window 91 displays a real-time state of the imaging subject, and displays identified anatomical key points on the subject 911 in an overlay manner. Optionally, the display window 91 may further display an identified state 92 of the subject in the overlay manner. For example, the state may be “moving in”, “moving out”, “occluded”, etc.

As an example, after the state of the subject is determined, automatic positioning processing and processing for automatically identifying an orientation of the subject may be triggered. For example, when it is determined that the state of the subject is “steadily positioned on a bed board”, the medical imaging system is automatically triggered to start positioning and identifying the orientation of the subject (the head facing a scanning center or the feet facing the scanning center). The positioning processing and the processing for identifying an orientation of the subject may be related technologies. The embodiments of the present application are not limited thereto. For example, for the positioning processing, the image capture apparatus acquires images containing the subject, detects coordinate positions (in a pixel coordinate system) of a site to be examined in the images, and determines, by estimation using a depth learning algorithm or a machine learning algorithm based on the type and coordinate positions of the site to be examined, that the site to be examined needs to be moved by a distance Z (in a coordinate system of the medical imaging system) before being aligned with the scanning center. Types and coordinate positions of sites to be examined of a plurality of volunteers positioned on a bed board may be acquired in advance as an input parameter set, and pre-measured or pre-calculated moving distances corresponding to the types and the coordinates of the sites to be examined may be used as an output parameter set. The depth learning algorithm is trained using the input parameter set and the output parameter set, and the distance Z by which the site to be examined needs to be moved is determined using the trained depth learning algorithm. For identification of the orientation of the subject, the orientation of the subject is also identified using the images obtained by the image capture apparatus by using an image recognition algorithm, which will not described again here.

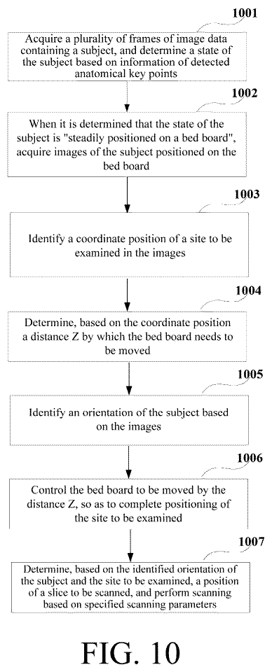

FIG. 10 is a schematic diagram of a scanning method according to embodiments of the present application. As shown in FIG. 10, at step 1001, the method includes acquiring a plurality of frames of image data containing a subject, and determining a state of the subject based on information of detected anatomical key points. The method also includes step 1002, where when it is determined that the state of the subject is “steadily positioned on a bed board”, images of the subject positioned on the bed board are acquired. At step 1003, a coordinate position of a site to be examined in the images is identified. Further at step 1004, a distance Z by which the bed board needs to be moved is determined based on the coordinate position.

Step 1005 of the method includes identifying an orientation of the subject based on the images and step 1006 includes controlling the bed board to be moved by the distance Z, so as to complete positioning of the site to be examined. Finally at step 1007, the method includes determining, based on the identified orientation of the subject and the site to be examined, a position of a slice to be scanned, and performing scanning based on specified scanning parameters.

In the foregoing embodiments, the order of execution of operations may be appropriately adjusted. In addition, some other operations may be added or some operations may be omitted. The present application is not limited thereto.

As an example, execution of an occlusion algorithm (for detecting a blanket and a coil) is automatically triggered when it is determined that the state of the subject is “partially occluded”; and with respect to the occlusion algorithm, the images obtained by the image capture apparatus are also used, and at least one of color channel information and depth information of the images is used to determine, using a depth learning algorithm, a body site covered by a blanket or a coil. For details, reference may be made to related technologies, and details will not be described here. Optionally, after the occlusion algorithm is executed, a key point position estimated using the depth learning algorithm may be displayed on an occluded position of the subject on the display window 91 in an overlay manner.

As an example, when it is determined that the state of the subject is “moving” and a moving direction is moving away from the bed board, or when it is determined that the subject is getting up, the user interface on the display of the medical imaging system is triggered to no longer display the anatomical key points of the subject, positioning and identification of an orientation of the subject are stopped, or execution of an occlusion algorithm is stopped. For example, once subject is lying steadily on the bed board, automatic positioning, identification of an orientation of the subject, execution of an occlusion algorithm, etc. are triggered. However, the subject may feel uncomfortable or encounter another situation, and the subject may get up to adjust a posture or even get out of the bed board and leave the scan room. When said states are detected, positioning and identification of an orientation of the subject are stopped, or execution of an occlusion algorithm is stopped, thereby avoiding faulty execution of the procedures.

As an example, when it is determined that the state of the subject is “falling”, an alert (a sound alert or an alert displayed on the user interface 90) is issued, the user interface on the display of the medical imaging system is triggered to no longer display the anatomical key points of the subject, positioning and identification of an orientation of the subject are stopped, or execution of an occlusion algorithm is stopped. The present application is not limited thereto. No further examples will be provided here.

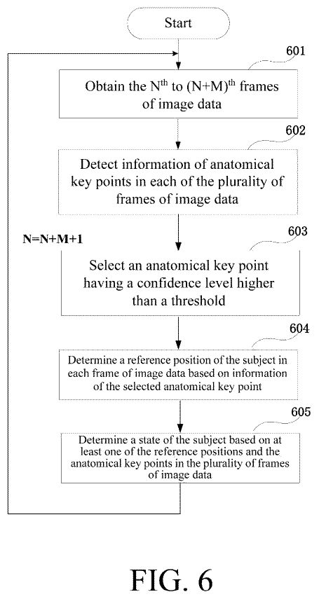

FIG. 6 is a schematic diagram of a subject tracking method according to embodiments of the present application. As shown in FIG. 6, the method includes a step 601 for Acquiring the N^{th} to (N+M)^{th} frames of image data containing a subject that are captured via an image capture apparatus and a step 602 for detecting information of anatomical key points in each of the plurality of frames of image data.

The method also includes a step 603 for selecting an anatomical key point having a confidence level higher than a threshold from the detected anatomical key points; a step 604 for determining a reference position of the subject in each frame of image data based on information of the selected anatomical key point. At step 605, the method includes determining a state of the subject based on at least one of the reference positions and the anatomical key points in the plurality of frames of image data, setting N to be equal to N+M+1, and returning to 601.

Implementations of 601 to 605 are as described above, and will not be described again here.

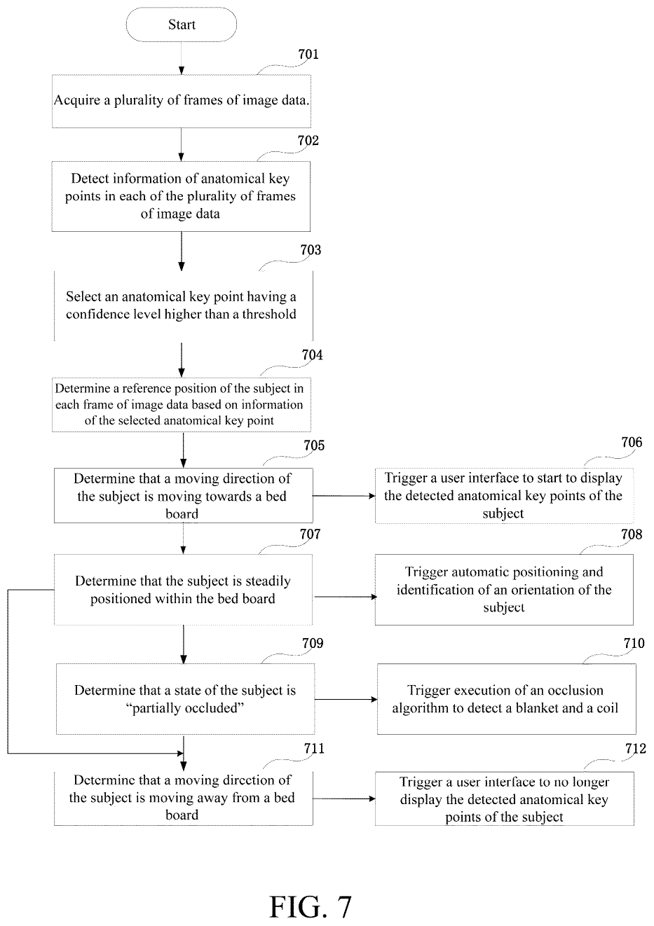

FIG. 7 is a schematic diagram of a medical imaging method for a subject according to embodiments of the present application. As shown in FIG. 7, for example, after the subject enters a scan room, the method includes the following steps:

701: Acquiring a plurality of frames of image data containing the subject, which are captured via an image capture apparatus;

702: Detecting information of anatomical key points in each of the plurality of frames of image data;

703: Selecting an anatomical key point having a confidence level higher than a threshold from the detected anatomical key points;

704: Determining a reference position of the subject in each frame of image data based on information of the selected anatomical key point;

705: Determining, based on the reference positions in the plurality of frames of image data, that a moving direction of the subject is moving towards a bed board;

706: Triggering a user interface on a display of a medical imaging system to start to display the detected anatomical key points of the subject;

707: Determining, based on the reference positions in the plurality of frames of image data, that the subject is steadily positioned within the bed board; and executing the determination of 709 and 711;

708: Triggering automatic positioning and identification of an orientation of the subject; and optionally, displaying positioning and orientation identification results on the user interface;

709: Determining, based on a change in the quantity of anatomical key points and a change in positions of the anatomical key points in the plurality of frames of image data, that a state of the subject is “partially occluded”;

710: Triggering execution of an occlusion algorithm to detect (positions, etc. of) a blanket and a coil;

711: Determining, based on the reference positions in the plurality of frames of image data, that the moving direction of the subject is moving away from the bed board; and

712: Triggering a user interface on a display of a medical imaging system to no longer display the detected anatomical key points of the subject.

In addition, in the process of 705 to 712, it may be further determined, based on at least one of a change in positions of the anatomical key points in the plurality of frames of image data, a change in a distance between anatomical key points, and a change in depths of the anatomical key points, that a pose of the subject is “getting up”, “lying down” or “falling”. When it is determined that the subject is falling, the medical imaging system may be further triggered to issue an alert. No further examples will be provided here.

It should be noted that the pluralities of frames of images in 705, 707, 709 and 711 are a different pluralities of frames of images or a the same plurality of frames of images. For example, in 705, the state of the subject is determined based on the N^{th} to the (N+M1)^{th} frames of images; in 707, the state of the subject is determined based on the (N+M1+1)^{th} to the (N+M1+M2)^{th} frames of images; in 709, the state of the subject is determined based on the (N+M1+M2+1)^{th} to the (N+M1+M2+M3)^{th} frames of images; and in 711, the state of the subject is determined based on the (N+M4)^{th} to the (N+M4+M5)^{th} frames of images, M1, M2, M3, M4 and M5 being integers greater than 1, and M1, M2, M3, M4 and M5 being the same or different. The embodiments of the present application are not limited thereto.

It should be noted that FIG. 2, FIG. 6, and FIG. 7 above merely schematically illustrate the embodiments of the present application, but the present application is not limited thereto. For example, the order of execution between operations may be appropriately adjusted. In addition, some other operations may be added or some operations may be omitted. Those skilled in the art could make appropriate variations according to the foregoing content, rather than being limited by the disclosures of FIG. 2, FIG. 6 and FIG. 7.

According to the described embodiments, a state of a subject is determined based on anatomical key points identified in images captured by an image capture apparatus. In this way, automatic tracking can be implemented after the subject enters a scan room, so that the determined state of the subject can be used as a condition for triggering other procedures in a scanning process, e.g., automatic positioning, automatic identification of an orientation, user interface display, execution of an occlusion algorithm, etc., thereby implementing further automation of the scanning process and improving scanning efficiency.

In addition, when it is detected that the subject gets up, gets out of bed or falls, a running procedure can be stopped in time. For example, the procedures for automatic positioning, automatic identification of an orientation, user interface display, and the execution of the occlusion algorithm can be stopped, thereby avoiding faulty execution of the procedures. In addition, an alert may be further issued to warn the clinical operator.

In addition, even when both the subject and the operator are located in the scan room, real-time tracking of the state of the subject can still assist in positioning or orientation identification processing, thereby providing more reference information for said processing. For example, the user interface is triggered to display more information.

Embodiments of the present application further provide a medical imaging system. FIG. 3 is a schematic diagram of a medical imaging system according to the embodiments of the present application. As shown in FIG. 3, the system 300 includes an image capture apparatus 301 for capturing a plurality of frames of image data containing a subject. The system 300 also includes a controller 302 connected to the image capture apparatus 301, and configured to: acquire a plurality of frames of image data containing the subject, which are captured via the image capture apparatus; detect information of anatomical key points in each of the plurality of frames of image data; and determine a state of the subject based on the information of the anatomical key points.

Regarding implementations of the image capture apparatus 301 and the controller 302, reference may be made to the foregoing embodiments, which will not be repeated here. The medical imaging system includes, but is not limited to, a computed tomography (CT) system, a magnetic resonance imaging (MRI) system, a C-arm imaging system, a positron emission computed tomography (PET) system, a single photon emission computed tomography (SPECT) system, an ultrasound system, an X-ray imaging system, or any other suitable medical imaging system.

In some embodiments, the controller 302 may be configured separately from a controller of the medical imaging system. For example, the controller 302 is configured as a chip or the like connected to the controller of the medical imaging system, and the two controllers may control each other. Alternatively, functions of the controller 302 may also be integrated into the controller of the medical imaging system. Embodiments of the present application are not limited thereto.

In some embodiments, the controller 302 includes a computer processor and a storage medium. Recorded on the storage medium is a program for predetermined data processing to be executed by the computer processor. For example, stored on the storage medium may be a program used to implement subject tracking for medical imaging. The storage medium may include, e.g., a ROM, a floppy disk, a hard disk, an optical disk, a magneto-optical disk, a CD-ROM, or a non-volatile memory card.

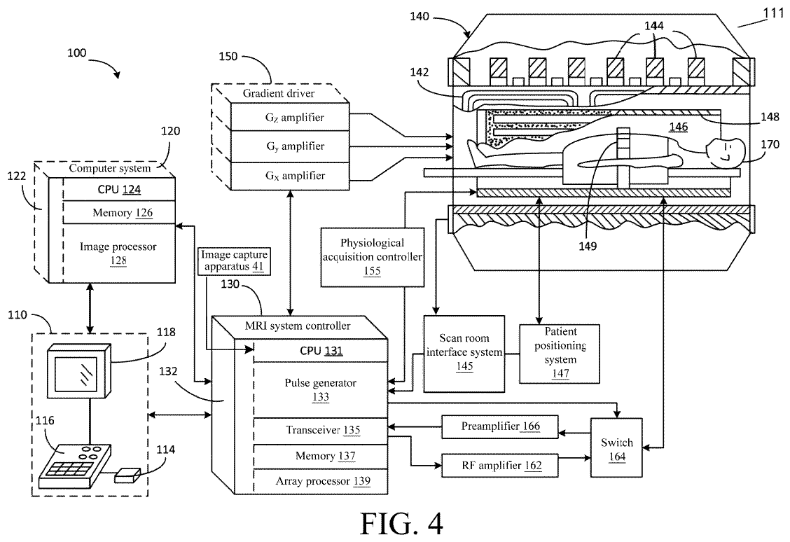

The medical imaging system may further include other structural components not shown in the figure. For details, references may be made to related technologies, and the embodiments of the present application are not limited thereto. Taking a magnetic resonance imaging system as an example, FIG. 4 is a schematic diagram of a magnetic resonance imaging system according to embodiments of the present application. As shown in FIG. 4, the difference relative to FIG. 1 is that the magnetic resonance imaging system further includes an image capture apparatus 41 that captures a plurality of frames of image data containing a subject. In addition, a controller 130 or a computer system 120 of the magnetic resonance imaging system is connected to the image capture apparatus 41 and is configured to: acquire the plurality of frames of image data containing the subject, which are captured via the image capture apparatus; detect information of anatomical key points in each of the plurality of frames of image data; and determine a state of the subject based on the information of the anatomical key points, and optionally after determining the state of the subject, perform at least one of the triggering a display change of a user interface of the medical imaging system, triggering automatic positioning processing, triggering processing for automatically identifying an orientation of the subject, triggering a blanket and coil occlusion algorithm, or issuing an alert. For a specific implementation of the magnetic resonance imaging system, reference may be made to the foregoing embodiments, which will not be repeated here.

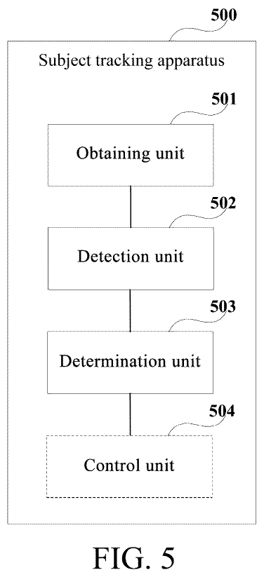

Embodiments of the present application further provide a subject tracking apparatus for medical imaging. FIG. 5 is a schematic diagram of the subject tracking apparatus for medical imaging according to the embodiments of the present application. As shown in FIG. 5, the apparatus 500 includes: an acquisition unit 501 for acquiring a plurality of frames of image data containing a subject, which are captured via an image capture apparatus. The apparatus 500 also includes a detection unit 502 for detecting information of anatomical key points in each of the plurality of frames of image data; and a determination unit 503 for determining a state of the subject based on the information of the anatomical key points.

For the implementations of modules of the foregoing units, reference may be made to 201 to 203, and repeated portions will not be described again here.

Optionally, the apparatus may further include: a control unit 504, which, after the determination unit 503 determines the state of the subject, performs at least one of controlling a display change of a user interface of a medical imaging system, controlling automatic positioning processing, controlling processing for automatically identifying an orientation of the subject, controlling execution of a blanket and coil occlusion algorithm, or controlling issuing of an alert.

Embodiments of the present application further provide a computer-readable program. The program, when executed in an apparatus or a medical imaging system, causes a computer to execute, in the apparatus or the medical imaging system, the subject tracking method for medical imaging described in the foregoing embodiments.

Embodiments of the present application further provide a storage medium having a computer-readable program stored therein. The computer-readable program causes a computer to execute, in an apparatus or a medical imaging system, the subject tracking method for medical imaging described in the foregoing embodiments.

Embodiments of the present application further provide a computer program product at least including a computer program/instructions. When a processor executes the computer program/instructions, the subject tracking method for medical imaging described in the foregoing embodiments is executed.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A subject tracking method for medical imaging, characterized in that the method comprises:
acquiring a plurality of frames of image data containing a subject, which are captured via an image capture apparatus;
detecting information of anatomical key points in each of the plurality of frames of image data; and
determining a state of the subject based on the information of the anatomical key points.

2. The method according to claim 1, wherein the image data comprises at least one of two-dimensional optical image data and depth image data.

3. The method according to claim 1, wherein the information of the anatomical key points comprises at least one of the following types of information: anatomical types of the key points, positional coordinates of the key points, confidence levels of the key points, and depth information of the key points.

4. The method according to claim 1, wherein the method further comprises:
selecting an anatomical key point having a confidence level higher than a threshold from the detected anatomical key points;
and determining the state of the subject based on information of the selected anatomical key point.

5. The method according to claim 1, wherein determining a state of the subject based on the information of the anatomical key points comprises:
determining a reference position of the subject in each frame of image data based on the information of the anatomical key points; and
determining, based on the reference positions in the plurality of frames of image data, that the state of the subject is “no movement” or “moving”.

6. The method according to claim 5, wherein the method further comprises:
determining, based on at least one of a result of comparison between the reference positions in the plurality of frames of image data and a standard position, or depth information of the reference positions in the plurality of frames of image data, a moving direction of the subject or a position of the subject when there is no movement.

7. The method according to claim 1, wherein determining a state of the subject based on the information of the anatomical key points comprises:
determining, based on a change in the quantity of anatomical keypoints and a change in positions of the anatomical key points in the plurality of frames of image data, whether the state of the subject is “partially occluded”.

8. The method according to claim 1, wherein determining a state of the subject based on the information of the anatomical key points comprises:
determining a posture change of the subject based on at least one of a change in position of an anatomical key point in the plurality of frames of image data, a change in a distance between anatomical key points, and a change in a depth of an anatomical key point.

9. The method according to claim 1, wherein the anatomical key points comprise: at least two of the top of the head, a shoulder, the nose, an eye, an ear, an arm, an elbow, a wrist, a hip, a knee, the heart, a pelvic cavity, the abdomen, the chest, and an ankle.

10. The method according to claim 1, wherein the method further comprises:
after determining the state of the subject, performing at least one of triggering a display change of a user interface of a medical imaging system, triggering automatic positioning processing, triggering processing for automatically identifying an orientation of the subject, triggering a blanket and coil occlusion algorithm, or issuing an alert.

11. A computer-readable storage medium, the computer-readable storage medium comprising a stored computer program, wherein the subject tracking method for medical imaging according to claim 1 is performed when the computer program is run.

12. A medical imaging system, characterized in that the system comprises:
an image capture apparatus, capturing a plurality of frames of image data containing a subject; and
a controller, connected to the image capture apparatus and used to execute the subject tracking method for medical imaging according to claim 1.
